(19) United States
(12) Patent Application Publication
(10) Pub. No.: US 2026/0028335 A1
(43) Pub. Date: Jan. 29, 2026

(54) NOVEL ISOTHIAZOL-3-YL AND ISOXAZOL-3-YL SULFONAMIDE COMPOUNDS

(71) Applicant: Hoffmann-La Roche Inc., Little Falls, NJ (US)

(72) Inventors: Guido GALLEY, Rheinfelden (DE); Luca Claudio GOBBI, Buus (CH); Wolfgang GUBA, Muellheim (DE); Dmitry MAZUNIN, Grenzach-Wyhlen (DE); Emmanuel PINARD, Linsdorf (FR); Antonio RICCI, Biel-Benken (CH)

(73) Assignee: Hoffmann-La Roche Inc., Little Falls, NJ (US)

(21) Appl. No.: 18/993,989

(22) PCT Filed: Jul. 18, 2023
(86) PCT No.: PCT/EP2023/069846

§ 371 Date: Jan. 13, 2025

(30) Foreign Application Priority Data
Jul. 20, 2022 (EP) 22185889.7
Aug. 2, 2022 (EP) 22188191.5

(51) Int. Cl. C07D 471/04; A61K 31/422; A61K 31/427; A61K 31/437; C07D 413/12; C07D 417/12

(52) U.S. Cl. CPC C07D 471/04; A61K 31/422; A61K 31/427; A61K 31/437; C07D 413/12; C07D 417/12

(57) ABSTRACT

The invention relates to novel compounds having the general formula (I) wherein R¹, R², R³, X and Y are as described herein, composition including the compounds and methods of using the compounds.

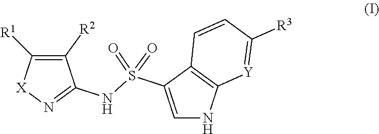

The present invention relates to organic compounds useful for therapy and/or prophylaxis in a mammal, and in particular to compounds that modulate GPR17 activity.

The present invention provides novel compounds of formula I

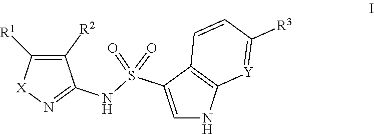

wherein,
R¹ is alkyl, alkoxy, cyano, cyanoalkyl, cyclopropyl, or halo;
R² is H, alkyl, alkoxy, cyano, cyclopropyl or halo;
R³ is halo, alkyl, or haloalkyl;
X is —O— or —S—;
Y is CH or N;
and pharmaceutically acceptable salts thereof.

Furthermore, the invention includes all racemic mixtures, all their corresponding enantiomers and/or optical isomers.

### BACKGROUND OF THE INVENTION

Myelination is a process that occurs robustly during development and despite the abundant presence of oligodendrocyte precursor cells (OPCs) throughout the adult CNS, the transition to myelinating oligodendrocytes and the production of restorative myelin sheaths around denuded axons is impaired in chronic demyelinating diseases. During development, myelination proceeds in a very orderly manner, with OPCs, characterized by expression of markers such as neural/glial antigen 2 (NG2) and platelet-derived growth factor alpha (PDGFRα), differentiating into oligodendrocytes which lose NG2 and PDGFRα expression and gain the expression of markers such as myelin basic protein (MBP) and myelin oligodendrocyte glycoprotein (MOG). The production of myelin by oligodendrocytes is a very tightly regulated process and in the CNS, this can be controlled by interactions with axons, well-understood in the peripheral but not in the central nervous system (Macklin, W. B. (2010). Sci. Signal. 3, pe32-pe32, “The myelin brake: When Enough Is Enough”). Myelination can also be controlled by internal brakes within oligodendrocytes themselves, through the transcription factor EB (TFEB)-PUMA axis or through GPR17 antagonism (Chen, Y., et al. (2009). Nat Neurosci 12, 1398-1406, “The oligodendrocyte-specific G protein-coupled receptor GPR17 is a cell-intrinsic timer of myelination”) (Sun, L. O., et al. (2018). Cell 175, 1811-1826.e21, “Spatiotemporal Control of CNS Myelination by Oligodendrocyte Programmed Cell Death through the TFEB-PUMA Axis”). Myelin serves not only to protect axons and facilitate neuronal transmission, but oligodendrocytes have also been shown to play an important role in metabolism of axons as well as in maintaining the electrolyte balance around axons (Schirmer, L., et al. (2014). Ann Neurol 75, 810-828, “Differential loss of KIR4.1 immunoreactivity in multiple sclerosis lesions”) (Simons, M., and Nave, K.-A. (2015). Cold Spring Harb Perspect Biol. 22, “Oligodendrocytes: Myelination and Axonal Support”).

GPR17 is a Class A orphan G protein-coupled receptor (GPCR). GPCRs are 7 domain transmembrane proteins that couple extracellular ligands with intracellular signaling via their intracellular association with small, heterotrimeric G-protein complexes consisting of G_{α}, G_{β}, G_{γ} subunits. It is the coupling of the GPCR to the G_{α} subunit that confers results in downstream intracellular signaling pathways. GPR17 is known to be coupled directly to G_{α i/o}, which leads to inhibition of adenylate cyclase activity, resulting in a reduction in cyclic AMP production (cAMP). GPR17 has also been shown to couple to G_{q/11}, that targets phospholipase C. Activation of phospholipase C leads to the cleavage of phosphatidylinositol 4,5-bisphosphate which produces inositol triphosphate (IP₃) and diacylglycerol (DAG). IP₃ consequently binds to the IP₃ receptor on the endoplasmic reticulum and causes an increase in intracellular calcium levels (Hanlon, C. D., and Andrew, D. J. (2015). J Cell Sci. 128, 3533-3542, “Outside-in signaling-a brief review of GPCR signaling with a focus on the Drosophila GPCR family”) (Inoue, A., et al. (2019), Cell 177, 1933-1947.e25, “Illuminating G-Protein-Coupling Selectivity of GPCRs”).

The role of GPR17 in myelination was first identified in a screen of the optic nerves of Olig1 knockout mice to identify genes regulating myelination. GPR17 expression was found to be expressed only in the myelinating cells of the CNS and absent from the Schwann cells, the peripheral nervous system's myelinating cells. The expression of GPR17 was found to be exclusively expressed in the oligodendrocyte lineage cells and was downregulated in myelinating oligodendrocyte (Chen, Y., et al. (2009)). Specifically, GPR17 expression is found to be present at low levels early on in the OPC and increases in the pre-myelinating oligodendrocyte before the expression is downregulated in the mature, myelinating oligodendrocyte (Boda, E., et al. (2011), Glia 59, 1958-1973, “The GPR17 receptor in NG2 expressing cells: Focus on in vivocell maturation and participation in acute trauma and chronic damage”) (Dziedzic, A., et al. (2020). Int. J. Mol. Sci. 21, 1852, “The gpr17 receptor—a promising goal for therapy and a potential marker of the neurodegenerative process in multiple sclerosis”) (Fumagalli, M. et al. (2011), J Biol Chem 286, 10593-10604, “Phenotypic changes, signaling pathway, and functional correlates of GPR17-expressing neural precursor cells during oligodendrocyte differentiation”). GPR17 knockout animals were shown to exhibit precocious myelination throughout the CNS and conversely, transgenic mice overexpressing GPR17 in oligodendrocytes with the CNP-Cre (2′, 3′-cyclic-nucleotide 3′-phosphodiesterase) promoter exhibited myelinogenesis defects, in line with what is to be expected of a cell-intrinsic brake on the myelination process (Chen, Y., et al. (2009)). Furthermore, loss of GPR17 enhances remyelination following demyelination with lysophosphatidylcholine-induced demyelination (Lu, C., Dong, et al. (2018), Sci. Rep. 8, 4502, “G-Protein-Coupled Receptor Gpr17 Regulates Oligodendrocyte Differentiation in Response to Lysolecithin-Induced Demyelination”). As such, antagonism of GPR17 that promotes the differentiation of oligodendrocyte lineage cells into mature, myelinating oligodendrocytes would lead to increase in myelination following demyelination.

Multiple sclerosis (MS) is a chronic neurodegenerative disease that is characterized by the loss of myelin, the protective fatty lipid layer surrounding axons, in the central nervous system (CNS). Prevention of myelin loss or remyelination of denuded axons is thought to prevent axonal degeneration and thus prevent progression of the disease (Franklin, R. J. (2002), Nat Rev Neurosci 3, 705-714, “Why does remyelination fail in multiple sclerosis?”). Due to the restorative impact that myelin repair has on the central nervous system, such a treatment will benefit all types of MS namely relapse-remitting, secondary progressive, primary progressive and progressive relapsing MS. Reparation of lost myelin will alleviate neurological symptoms associated with MS due to the neuroprotective effect of preserving axons.

Due to the essential role that myelination plays in functioning of the nervous system, facilitating OPC to oligodendrocyte differentiation has the potential to impact multiple diseases where white matter defects/irregularities due to either loss of myelinating oligodendrocytes or hampered differentiation of OPCs to oligodendrocytes have been observed, due to the disease itself or inflammation. This is in addition to the diseases where GPR17 expression itself is altered.

The diseases that GPR17 antagonism can be thus used to yield a positive disease outcome include, but are not limited to:
Direct damage to myelin sheaths:
   Metabolic conditions that lead to destruction of central myelin such as central pontine myelinolysis, extra-pontine myelinolysis due to overly-rapid correction of hyponatremia in conditions for instance, but not limited to, alcoholism, liver disease, immunosuppression after transplantation
   Carbon monoxide poisoning where oligodendrocyte dysfunction and failure to regenerate has been reported in the deep white matter layers of the brain
   Nutritional deficiency that results in myelin loss or failure to properly generate myelin during development
   Virus-induced demyelination
Primary demyelinating disorders
   Multiple Sclerosis (relapse-remitting, secondary progressive, primary progressive and progressive relapsing MS)
   Acute and multiphasic disseminated encephalomyelitis
   Neuromyelitis optica spectrum disorders including optic neuritis
   Transverse myelitis
   Leukodystrophies such as adrenoleukodystrophy, adrenomyeloneuropathy and other inherited leukodystrophies that result in myelin loss
CNS disorders with associated myelin loss:
   Alzheimer's Disease
   Schizophrenia
   Parkinson's Disease
   Huntington's disease
   Amyotrophic lateral
   Ischemia due to stroke
Other diseases:
   Inflammation in the CNS for instance following encephalitis, primary angiitis, meningitis

The compounds of formula I bind to and modulates GPR17 activity.

The compounds of formula I are therefore particularly useful in the treatment of diseases related to GPR17 antagonism.

The compounds of formula I are particularly useful in the treatment or prophylaxis of multiple sclerosis (MS), conditions related to direct damage to myelin sheaths such as carbon monoxide poisoning or virus induced demyelination, primary demyelinating disorders such as acute and multiphasic disseminated encephalomyelitis, and other CNS disorders associated with myelin loss such as Alzheimer's disease, schizophrenia, Parkinson's disease and Huntington's disease.

### SUMMARY OF THE INVENTION

The present invention provides novel compounds of formula I

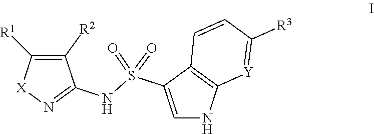

wherein,
   R¹ is alkyl, alkoxy, cyano, cyanoalkyl, cyclopropyl, or halo;
   R² is H, alkyl, alkoxy, cyano, cyclopropyl or halo;
   R³ is halo, alkyl, or haloalkyl;
   X is —O— or —S—;
   Y is CH or N;
   and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl, ethyl and propyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular examples are methoxy and ethoxy.

The term “cyano” denotes a —C—N group.

“Cyanoalkyl” means a moiety of the formula —R′—R″, where R′ is alkyl as defined herein and R″ is cyano or nitrile. Particular example is cyanoethyl.

The term “halogen”, “halide” and “halo” are used interchangeably herein and denote fluoro, chloro, bromo or iodo. Particular halogens are chloro and bromo.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is alkyl, alkoxy, cyanoalkyl or cyclopropyl.

An embodiment of the present invention provides compounds according to formula I as described herein, wherein R² is H, alkyl, alkoxy, cyano, cyclopropyl or halo.

An embodiment of the present invention provides compounds according to formula I as described herein, wherein R² is alkoxy or halo.

An embodiment of the present invention provides compounds according to formula I as described herein, wherein R³ is halo or alkyl.

An embodiment of the present invention provides compounds according to formula I as described herein, wherein R³ is halo.

An embodiment of the present invention provides compounds according to formula I as described herein, wherein X is —S—.

An embodiment of the present invention provides compounds according to formula I as described herein, wherein
R¹ is alkyl, alkoxy, cyanoalkyl, or cyclopropyl;
R² is H, alkyl, alkoxy, cyano, cyclopropyl or halo;
R³ is halo;
X is —O— or —S—;
Y is CH or N;
and pharmaceutically acceptable salts thereof.

An embodiment of the present invention provides compounds according to formula I as described herein, wherein
R¹ is alkyl, alkoxy, cyanoalkyl, or cyclopropyl;
R² is alkoxy or halo;
R³ is halo;
X is —S—;
Y is CH or N;
and pharmaceutically acceptable salts thereof.

Particular examples of compounds of formula I as described herein are selected from
6-chloro-N-(4-chloro-5-propyl-isothiazol-3-yl)-1H-indole-3-sulfonamide;
6-chloro-N-(4-chloro-5-cyclopropyl-isothiazol-3-yl)-1H-indole-3-sulfonamide;
6-chloro-N-(4-chloro-5-cyclopropyl-isothiazol-3-yl)-1H-pyrrolo[2,3-b]pyridine-3-sulfonamide;
6-bromo-N-(4-chloro-5-cyclopropyl-isothiazol-3-yl)-1H-pyrrolo[2,3-b]pyridine-3-sulfonamide;
6-chloro-N-(4-chloro-5-methyl-isothiazol-3-yl)-1H-indole-3-sulfonamide;
6-chloro-N-(4,5-dimethylisothiazol-3-yl)-1H-indole-3-sulfonamide;
6-chloro-N-(5-ethyl-4-methyl-isoxazol-3-yl)-1H-indole-3-sulfonamide;
6-chloro-N-(4-chloro-5-ethyl-isothiazol-3-yl)-1H-indole-3-sulfonamide;
6-chloro-N-(4-ethyl-5-methyl-isothiazol-3-yl)-1H-indole-3-sulfonamide;
6-chloro-N-(4-chloro-5-methoxy-isothiazol-3-yl)-1H-indole-3-sulfonamide;
6-chloro-N-(4-chloro-5-ethoxy-isothiazol-3-yl)-1H-indole-3-sulfonamide;
6-chloro-N-[4-chloro-5-(2-cyanoethyl)isothiazol-3-yl]-1H-indole-3-sulfonamide;
6-chloro-N-(5-methylisothiazol-3-yl)-1H-indole-3-sulfonamide;
6-chloro-N-(4-cyclopropyl-5-methyl-isothiazol-3-yl)-1H-indole-3-sulfonamide;
6-chloro-N-(4-cyano-5-methyl-isothiazol-3-yl)-1H-indole-3-sulfonamide;
6-chloro-N-(4-methoxy-5-methyl-isothiazol-3-yl)-1H-indole-3-sulfonamide;
6-chloro-N-(4-methoxy-5-propyl-isothiazol-3-yl)-1H-indole-3-sulfonamide;
6-chloro-N-(4-methyl-5-propyl-isoxazol-3-yl)-1H-indole-3-sulfonamide;
6-chloro-N-(4,5-dimethylisoxazol-3-yl)-1H-indole-3-sulfonamide;
6-chloro-N-(4-chloro-5-methyl-isoxazol-3-yl)-1H-indole-3-sulfonamide;
N-(4-bromo-5-methyl-isoxazol-3-yl)-6-chloro-1H-indole-3-sulfonamide;
and pharmaceutically acceptable salts thereof.

Preferred examples of compounds of formula I as described herein are selected from
6-chloro-N-(4-chloro-5-propyl-isothiazol-3-yl)-1H-indole-3-sulfonamide;
6-chloro-N-(4-chloro-5-cyclopropyl-isothiazol-3-yl)-1H-indole-3-sulfonamide;
6-bromo-N-(4-chloro-5-cyclopropyl-isothiazol-3-yl)-1H-pyrrolo[2,3-b]pyridine-3-sulfonamide;
6-chloro-N-(4-chloro-5-methyl-isothiazol-3-yl)-1H-indole-3-sulfonamide;
6-chloro-N-(4-chloro-5-ethyl-isothiazol-3-yl)-1H-indole-3-sulfonamide;
6-chloro-N-(4-chloro-5-ethoxy-isothiazol-3-yl)-1H-indole-3-sulfonamide;
6-chloro-N-[4-chloro-5-(2-cyanoethyl)isothiazol-3-yl]-1H-indole-3-sulfonamide;
6-chloro-N-(4-methoxy-5-propyl-isothiazol-3-yl)-1H-indole-3-sulfonamide;
   and pharmaceutically acceptable salts thereof.

Processes for the manufacture of compounds of formula I as described herein are an object of the invention.

The present compounds of formula I and their pharmaceutically acceptable salts can be prepared by methods known in the art, for example, by processes described below, which process comprises reacting a compound of formula III with a compound of formula II in the presence of a base selected from N-ethyldiisopropylamine or pyridine with or without the addition of the catalyst 4-dimethylaminopyridine, or in the presence of an inorganic base like potassium phosphate, to provide a compound of formula I,

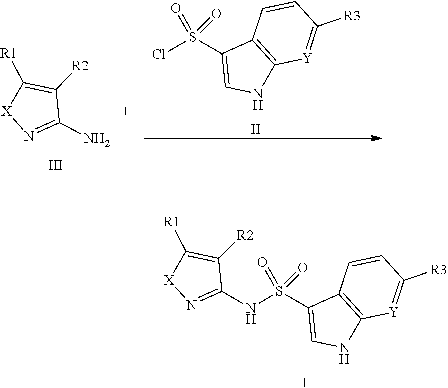

wherein R¹, R², R³, X and Y are as described above.

### General Synthetic Schemes

The compounds of formula I may be prepared in accordance with the process variant described above and with the following scheme 1. The starting materials are commercially available or may be prepared in accordance with known methods.

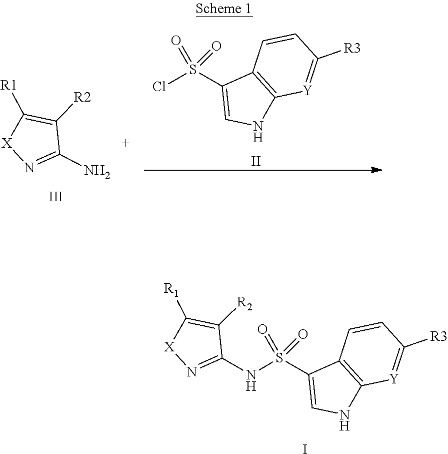

Compounds of general formula I can be prepared by reacting sulfonylchloride II with amines III in the presence of a base like N-ethyldiisopropylamine or pyridine with or without addition of a catalytic amount of 4-dimethylaminopyridine, or in presence of an inorganic base like potassium phosphate. The starting materials are commercially available or may be prepared in accordance with known methods (see Scheme 1).

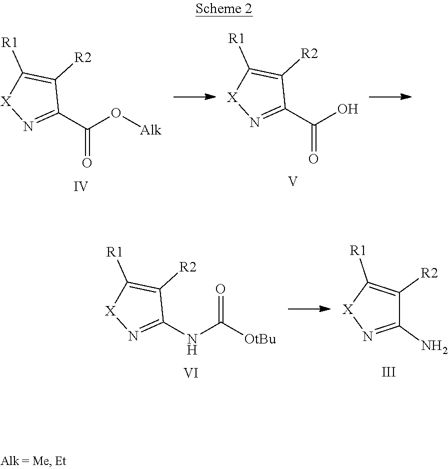

Amines of formula III can be prepared by saponification of ester IV with a base such as sodium hydroxide or lithium hydroxide to give acid V. Reaction of acid V with diphenylphosphoryl azide in presence of tert-butanol leads to intermediate VI which can be deprotected by treatment with an acid such as hydrochlorid acid or trifluoroacetic acid to yield amine III (see Scheme 2).

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

The invention also relates in particular to:
A compound of formula I for use as therapeutically active substance;
A compound of formula I for use in the treatment of a disease modulated by GPR17;

Likewise an object of the present invention is a pharmaceutical composition comprising a compound according to formula I as described herein and a therapeutically inert carrier.

The use of a compound of formula I for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the treatment or prophylaxis of multiple sclerosis, Alzheimer's disease, Parkinson's disease, or Huntington's disease.

A particular embodiment of the invention is the use of a compound of formula I for the treatment or prophylaxis of multiple sclerosis.

The use of a compound of formula I for the preparation of a medicament for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the preparation of a medicament for the treatment or prophylaxis of multiple sclerosis, Alzheimer's disease, Parkinson's disease, or Huntington's disease.

A particular embodiment of the invention is the use of a compound of formula I for the preparation of a medicament for the treatment or prophylaxis of multiple sclerosis.

A compound according to formula I for use in the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is a compound of formula I for use in the treatment or prophylaxis of multiple sclerosis, Alzheimer's disease, Parkinson's disease, or Huntington's disease.

A particular embodiment of the invention is a compound according to formula I for use in the treatment or prophylaxis of multiple sclerosis.

A method for the treatment or propylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity, which method comprises administering an effective amount of a compound of formula I to a patient in need thereof.

An embodiment of the present invention is a method for the treatment or prophylaxis of multiple sclerosis, Alzheimer's disease, Parkinson's disease, or Huntington's disease, which method comprises administering an effective amount of a compound of formula I to a patient in need thereof.

A particular embodiment of the invention is a method for the treatment or prophylaxis of multiple sclerosis, which method comprises administering an effective amount of a compound of formula I to a patient in need thereof.

Also an embodiment of the present invention provides compounds of formula I as described herein, when manufactured according to any one of the described processes.

### Assay Procedures

GPR17 cAMP Assay Protocol:

CHO-K1 cells stably expressing vector containing untagged human GPR17 short isoform (Roche) were cultured at 37° C./5% CO2 in DMEM (Dulbecco's Modified Eagle Medium):F-12 (1:1) supplemented with 10% foetal bovine serum and 400 μg/ml Geneticin.

Changes in intracellular cyclic adenosine monophosphate (cAMP) levels were quantified using the Nano-TRF Detection Assay kit (Roche Diagnostics, Cat. No. 05214386001). This assay allows for direct cAMP quantification in a homogeneous solution. cAMP is detected based on time-resolved fluorescence energy transfer (TR-FRET) and competitive binding of ruthenylated cAMP and endogenous cAMP to an anti-cAMP monoclonal antibody labeled with AlexaFluor-700. The Ruthenium complex serves as the FRET donor and transfers energy to AlexaFluor-700. The FRET signal is inversely proportional to the cAMP concentration.

CHO-GPR17S cells were detached with Accutase and resuspended in assay buffer consisting of Hank's Balanced Salt Solution (HBSS), 10 mM HEPES (4-(2-hydroxyethyl) piperazine-1-ethanesulfonic acid solution) and 0.1% bovine serum albumin (pH 7.4). The cells were seeded in black 384-well plates (Corning) at a density of 10,000 cells/20 μl assay buffer until the addition of compounds.

Test antagonist compounds were serially diluted in dimethyl sulfoxide (DMSO) and spotted in 384-well plates. The compounds were then diluted in HBSS buffer supplemented with an EC80 concentration of MDL29,951 (3-(2-Carboxy-4,6-dichloroindol-3-yl)propionic acid) (GPR17 agonist) plus 3-Isobutyl-1-methylxanthine (IBMX) (0.5 mM final concentration) and added to the cells at room temperature. Forskolin (15 μM final concentration) was added 5 minutes after the test compounds and the cells were incubated at room temperature for 30 minutes. The assay was stopped by adding cAMP detection mix (containing detergents for cell lysis) for 90 minutes at room temperature.

Cellular cAMP was measured using a Paradigm reader (Molecular Devices). The raw data was used to calculate the FRET signal based on the assay's P-factor as per cAMP kit instructions. The data was normalized to the maximal activity of a reference antagonist and dose response curves were fitted to the percent activity of the test compounds using a sigmoidal dose response model (Genedata Screener).

Results in the hGPR17 cAMP assay are provided for compounds of formula I in Table 1

TABLE 1

| Example number | hGPR17 cAMP IC50 [μM] |
|---|---|
| 1 | 0.019 |
| 2 | 0.051 |
| 3 | 0.131 |
| 4 | 0.103 |
| 5 | 0.106 |
| 6 | 0.216 |
| 7 | 0.452 |
| 8 | 0.105 |
| 9 | 0.659 |
| 10 | 0.113 |
| 11 | 0.054 |
| 12 | 0.074 |
| 13 | 0.629 |
| 14 | 1.110 |
| 15 | 0.499 |
| 16 | 0.857 |
| 17 | 0.078 |
| 18 | 0.815 |
| 19 | 0.550 |
| 20 | 0.404 |
| 21 | 0.315 |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen atmosphere if not specified otherwise.

### Intermediates A

### Intermediate A1: 6-chloro-1H-indole-3-sulfonyl Chloride

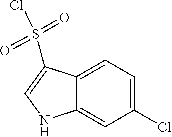 [SMILES: O=S(=O)(Cl)c1c[nH]c2cc(Cl)ccc12]

Intermediate A1 is commercial (CAS 1216060-79-5)

### Intermediate A2: 6-chloro-1H-pyrrolo[2,3-b]pyridine-3-sulfonyl Chloride

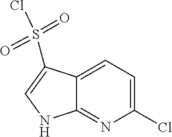 [SMILES: O=S(=O)(Cl)c1c[nH]c2nc(Cl)ccc12]

Intermediate A2 is known (CAS 2231234-21-0) and was synthesized according to WO2018/122232.

### Intermediate A3: 6-bromo-1H-pyrrolo[2,3-b]pyridine-3-sulfonyl Chloride

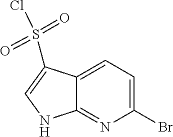 [SMILES: O=S(=O)(Cl)c1c[nH]c2nc(Br)ccc12]

Intermediate A3 is known (CAS 2231234-27-6) and was synthesized according to WO2018/122232.

### Intermediates B

### Intermediate B1: 4-chloro-5-propyl-isothiazol-3-amine Hydrochloride

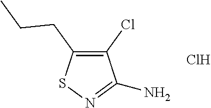

### Step 1: 4-chloro-5-prop-1-enyl-isothiazole-3-carboxylic Acid Methyl Ester

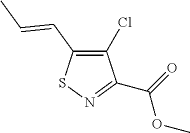

A reaction mixture of 4,5-dichloroisothiazole-3-carboxylic acid methyl ester (CAS 166668-76-4, 200 mg, 0.94 mmol), 4,4,5,5-tetramethyl-2-(prop-1-en-1-yl)-1,3,2-dioxaborolane (792 mg, 4.72 mmol), 1,1′-bis(diphenylphosphino)ferrocene-palladium(II)dichloride dichloromethane complex (39 mg, 0.047 mmol) and potassium carbonate (521 mg, 3.77 mmol) in 1,4-dioxane (4 ml) and water (0.5 ml) was stirred at 90° C. for 3 h. The reaction mixture was diluted with water and extracted with ethyl acetate (3×5 ml). The combined organic layers were washed with water, brine, dried over sodium sulfate, filtered and concentrated to dryness. The residue was purified by flash chromatography (silica gel, 0-20% ethyl acetate in heptane) to afford 4-chloro-5-prop-1-enyl-isothiazole-3-carboxylic acid methyl ester (114 mg, 53% yield) as light yellow solid. MS m/z: 218.0 [M+H]⁺, ESI pos.

### Step 2: 4-chloro-5-propyl-isothiazole-3-carboxylic Acid Methyl Ester

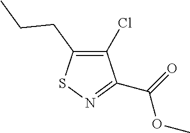

A solution of 4-chloro-5-prop-1-enyl-isothiazole-3-carboxylic acid methyl ester (114 mg, 0.50 mmol) in methanol (3 ml) was purged with argon and evacuated under vacuo (3×). Then platinum (IV) oxide (7 mg, 0.025 mmol) was added and the evacuation was repeated. The reaction mixture was stirred at room temperature for 16 h under a hydrogen atmosphere. The reaction mixture was filtered through Celite and concentrated to dryness to afford 4-chloro-5-propyl-isothiazole-3-carboxylic acid methyl ester (33 mg, 24%) as off-white liquid. MS m/z: 220.1 [M+H]⁺, ESI pos.

### Step 3: 4-chloro-5-propyl-isothiazole-3-carboxylic Acid

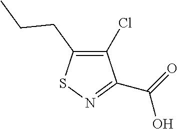

To a solution of 4-chloro-5-propyl-isothiazole-3-carboxylic acid methyl ester (33 mg, 0.15 mmol) in tetrahydrofuran (0.5 ml) was added sodium hydroxide solution (1 M in water, 179 ul, 0.179 mmol). The reaction mixture was stirred at room temperature for 1 h, then acidified with 1M hydrochloric acid and extracted with ethyl acetate (3×10 ml). The combined organic layers were washed with water, brine, dried over sodium sulfate, filtered and concentrated to afford 4-chloro-5-propyl-isothiazole-3-carboxylic acid (37 mg, 98% yield) as light brown solid. MS m/z: 206.1 [M+H]⁺, ESI pos.

### Step 4: tert-butyl N-(4-chloro-5-propyl-isothiazol-3-yl)carbamate

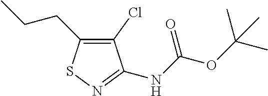 [SMILES: CCCc1snc(NC(=O)OC(C)(C)C)c1Cl]

To a solution of 4-chloro-5-propyl-isothiazole-3-carboxylic acid (37.5 mg, 0.145 mmol) in tert-butanol (0.5 ml) were added diphenyl phosphoryl azide (44 mg, 34 ul, 0.160 mmol) and triethylamine (29 mg, 41 μL, 0.29 mmol). The reaction mixture was stirred at 90° C. for 2 h. After cooling down to room temperature the reaction mixture was diluted with water and extracted with ethyl acetate (3×10 ml). The combined organic layers were washed with water, brine, dried over sodium sulfate, filtered and concentrated to dryness. The crude compound was purified by flash chromatography (silica gel, 0-25% ethyl acetate in heptane) to afford tert-butyl N-(4-chloro-5-propyl-isothiazol-3-yl)carbamate (37 mg, 83% yield) as colorless oil. 221.0 [M−isobutene+H]⁺, ESI pos.

### Step 5: 4-chloro-5-propyl-isothiazol-3-amine Hydrochloride

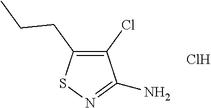

A solution of tert-butyl N-(4-chloro-5-propyl-isothiazol-3-yl)carbamate (37 mg, 0.134 mmol) in dichloromethane (1 ml) was treated with 4M hydrochloric acid (202 mg, 168 ul, 0.670 mmol) and the mixture was stirred at 23° C. for 1 h. The reaction mixture was evaporated to dryness to afford the title compound (20.5 mg, 61%) as white powder. MS m/z: 176.9 [M+H]⁺, ESI pos.

### Intermediate B2: 4-methoxy-2-propyl-pyrimidin-5-amine Hydrochloride

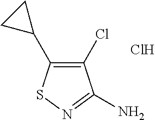

### Step 1: methyl 4-chloro-5-cyclopropyl-isothiazole-3-carboxylate

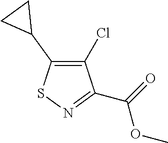

A reaction mixture of 4,5-dichloroisothiazole-3-carboxylic acid methyl ester (CAS 166668-76-4, 200 mg, 0.943 mmol), potassium cyclopropyl(trifluoro)borate (698 mg, 4.72 mmol), palladium (II) acetate (11 mg, 0.047 mmol), di(adamantan-1-yl)(butyl)phosphine (17 mg, 0.047 mmol) and cesium carbonate (922 mg, 2.83 mmol) in toluene (3.5 ml) and water (0.35 ml) was stirred at 100° C. for 16 h. The reaction mixture was diluted with water and extracted with ethyl acetate (2×20 ml). The combined organic layers were washed with water, brine, dried over sodium sulfate, filtered and concentrated to dryness. The residue was purified by flash chromatography (silica gel, 0-25% ethyl acetate in heptane) to afford methyl 4-chloro-5-cyclopropyl-isothiazole-3-carboxylate (111 mg, 51% yield) as light yellow oil. MS m/z: 218.0 [M+H]⁺, ESI pos.

### Step 2: 4-chloro-5-cyclopropyl-isothiazole-3-carboxylic Acid

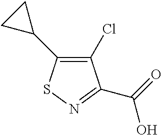 [SMILES: O=C(O)c1nsc(C2CC2)c1Cl]

To a solution of methyl 4-chloro-5-cyclopropyl-isothiazole-3-carboxylate (111 mg, 0.484 mmol) in tetrahydrofuran (2 ml) was added 1 M aqueous sodiumhydroxide solution (581 ul, 0.581 mmol). The reaction mixture was stirred at room temperature for 1 h. The reaction mixture was acidified with 1M aqueous hydrochloric acid and extracted with ethyl acetate (3×10 ml). The combined organic layers were washed with water, brine, dried over sodium sulfate, filtered and concentrated to afford 4-chloro-5-cyclopropyl-isothiazole-3-carboxylic acid (97 mg, 97%) as light brown powder. MS m/z: 204.0 [M+H]⁺, ESI pos.

### Step 3: tert-butyl N-(4-chloro-5-cyclopropyl-isothiazol-3-yl)carbamate

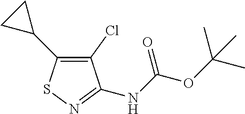 [SMILES: CC(C)(C)OC(=O)Nc1nsc(C2CC2)c1Cl]

To a solution of 4-chloro-5-cyclopropyl-isothiazole-3-carboxylic acid (97 mg, 0.471 mmol) in tert-butanol (3 ml) were added diphenylphosphoryl azide (142 mg, 111 ul, 0.518 mmol) and triethylamine (95 mg, 131 ul, 0.941 mmol). The reaction mixture was stirred at 90° C. for 2 h and was cooled down to room temperature. The reaction mixture was diluted with water and extracted with ethyl acetate (3×10 ml). The combined organic layers were washed with water, brine, dried over sodium sulfate, filtered and concentrated to dryness. The residue was purified by flash chromatography (silica gel, 0-25% ethyl acetate in heptane) to afford tert-butyl N-(4-chloro-5-cyclopropyl-isothiazol-3-yl)carbamate (108 mg, 80%) as white powder. 1H NMR (300 MHz, CDCl₃) δ ppm 0.78-0.87 (m, 2H) 1.14-1.23 (m, 2H) 1.54 (s, 9H) 2.08-2.18 (m, 1H) 7.12 (br s, 1H).

### Step 4: 4-methoxy-2-propyl-pyrimidin-5-amine Hydrochloride

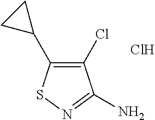

A solution of tert-butyl N-(4-chloro-5-cyclopropyl-isothiazol-3-yl)carbamate (95 mg, 0.328 mmol) in dichloromethane (1 ml) was treated with 4 M aqueous hydrochloric acid (493 mg, 411 ul, 1.64 mmol) and stirred at room temperature for 1 h. The reaction mixture was evaporated to dryness to afford (4-chloro-5-cyclopropyl-isothiazol-3-yl)amine hydrochloride (76 mg, 99%) as white powder. MS m/z: 175.0 [M+H]⁺, ESI pos.

### Intermediate B3: 4-chloro-5-methyl-isothiazol-3-amine

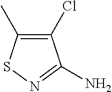

### Step 1: methyl 4,5-dichloroisothiazole-3-carboxylate

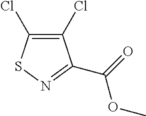

To a solution of 4,5-dichloroisothiazole-3-carboxylic acid (CAS 131947-13-2, 400 mg, 2.02 mmol) in tetrahydrofuran (10 ml) and methanol (1 ml) under argon, at 0° C., was added 2 M diazomethyl(trimethyl)silane in diethylether (1.5 ml, 3.0 mmol) dropwise within 5 min. The light yellow solution was stirred at room temperature for 15 min. The reaction was cooled to 0° C. and quenched with a solution of acetic acid and water (1:1, 0.4 ml). The yellow solution was stirred at 0° C. for 30 min and 15 min at room temperature. Water was added and the aqueous phase was extracted with ethyl acetate (3×). The combined organic layers were washed with brine, dried over sodium sulfate, filtered and concentrated in vacuo. The residue was purified by flash chromatography (silica gel, 0-20% ethyl acetate in heptane) to obtain methyl 4,5-dichloroisothiazole-3-carboxylate (395 mg, 92% yield) as a white solid. MS m/z: 212.0 [M+H]⁺, ESI pos.

### Step 2: 4-chloro-5-methyl-isothiazole-3-carboxylic Acid

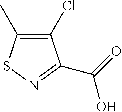

To a suspension of methyl 4,5-dichloroisothiazole-3-carboxylate (310 mg, 1.45 mmol), methylboronic acid (893 mg, 14.5 mmol) and potassium carbonate (2.0 g, 14.5 mmol) in a mixture of 1,4-dioxane (6 ml) and water (0.6 ml) was added 1,1′-bis(diphenylphosphino)ferrocene-palladium(II)dichloride dichloromethane complex (120 mg, 0.145 mmol). The orange suspension was stirred at 90° C. for 75 min, then allowed to cool to room temperature. It was diluted with water and ethyl acetate. The mixture was acidified with hydrochloric acid and extracted with ethyl acetate (3×). The organic layers were dried over sodium sulfate, filtered and concentrated in vacuo to afford crude 4-chloro-5-methyl-isothiazole-3-carboxylic acid (185 mg, 45% purity, 29% yield) which was directly used for the next step, orange solid. MS m/z: 178.1 [M+H]⁺, ESI pos.

### Step 3: 4-chloro-5-methyl-isothiazol-3-amine

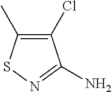

To a solution of 4-chloro-5-methyl-isothiazole-3-carboxylic acid (185 mg, 45% purity, 0.469 mmol) in tert-butanol (1 ml) was added triethylamine (95 mg, 131 ul, 0.937 mmol) and diphenylphosphoryl azide (142 mg, 111 ul, 0.516 mmol). After stirring at 90° C. overnight, more triethylamine (47 mg, 65 ul, 0.468 mmol) and diphenylphosphoryl azide (129 mg, 100 ul, 0.468 mmol) was added and stirring was continued for 2 h, then the reaction was allowed to cool to room temperature and concentrated in vacuo. The reaction was diluted with saturated sodium bicarbonate solution and extracted 3 times with ethyl acetate. The combined organic layers were dried over sodium sulfate, filtered and concentrated in vacuo. The residue was purified by flash chromatography (silica gel, 0-20% ethyl acetate in heptane) to give tert-butyl N-(4-chloro-5-methyl-isothiazol-3-yl)carbamate (20 mg, 17% yield) and 4-chloro-5-methyl-isothiazol-3-amine (16 mg, 23% yield) as white solids.

To obtain a second batch of the title compound, tert-butyl N-(4-chloro-5-methyl-isothiazol-3-yl)carbamate (20 mg, 0.081 mmol) was dissolved in 1,2-dichloroethane (0.17 ml) and trifluoroacetic acid (46 mg, 31 ul, 0.404 mmol). After 8 h stirring at room temperature the reaction was quenched with saturated sodium bicarbonate solution and extracted with dichloromethane (3×). The combined organic layers were dried over sodium sulfate, filtered and concentrated in vacuo to give the title compound (9 mg, 73% yield) as an off white solid. MS m/z: 149.1 [M+H]⁺, ESI pos.

### Intermediate B4: 4,5-dimethylisothiazol-3-amine

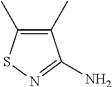

### Step 1: tert-butyl N-(5-methylisothiazol-3-yl)carbamate

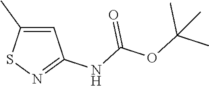

To a solution of 5-methylisothiazole-3-carboxylic acid (300 mg, 2.1 mmol) and triethylamine (233 mg, 322 ul, 2.31 mmol) in tetrahydrofuran (9 ml) was added dropwise at −10° C. ethyl chloroformate (250 mg, 220 ul, 2.31 mmol). The mixture was stirred at −10° C. for 30 min. Then a solution of sodium azide (682 mg, 10.5 mmol) in water (3 ml) was added slowly added, the mixture was allowed to warm to room temperature and stirred for 1 h. The mixture was diluted with ice/water (20 ml) and extracted with dichloromethane (3×20 ml). The combined organic layers were dried over sodium sulfate, filtered and concentrated in vacuo. The residue was dissolved in toluene (9 ml), tert-butanol (4.5 ml) was added and the mixture was heated to 80° C. for 3.5 h. The mixture was concentrated in vacuo. The residue was purified by flash column chromatography (silica gel, 0-30% ethyl acetate in heptane) to give tert-butyl N-(5-methylisothiazol-3-yl)carbamate (233 mg, 52% yield) as white solid. MS m/z: 159.0 [M−isobutene+H]⁺, ESI pos.

### Step 2: tert-butyl N-(4-bromo-5-methyl-isothiazol-3-yl)carbamate

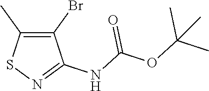

To a solution of tert-butyl N-(5-methylisothiazol-3-yl)carbamate (203 mg, 0.947 mmol) in acetic acid (2.6 ml) was added at 22° C. sodium acetate (256 mg, 3.13 mmol) followed by bromine (332 mg, 108 ul, 2.08 mmol). The tube was sealed and the mixture was stirred at 50° C. for 1.5 h.

After cooling the mixture was poured into cold aqueous 10% sodium carbonate solution (60 ml). The aqueous layer was extracted with ethyl acetate (2×50 ml), then the combined organic layers were dried over sodium sulfate, filtered and concentrated in vacuo.

The residue was purified by flash column chromatography (silica gel, 0-30% ethyl acetate in heptane) to give tert-butyl N-(4-bromo-5-methyl-isothiazol-3-yl)carbamate (128 mg, 46% yield) as light yellow oil. MS m/z: 237.0, 239.0 [M+H]⁺, ESI pos.

### Step 3: tert-butyl N-(4,5-dimethylisothiazol-3-yl)carbamate

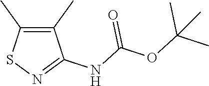

To a solution of tert-butyl N-(4-bromo-5-methyl-isothiazol-3-yl)carbamate (63 mg, 0.215 mmol) in 1,4-dioxane (1.3 ml) was added at 22° C. trimethylboroxine (67 mg, 75 ul, 0.537 mmol) followed by potassium carbonate (119 mg, 0.86 mmol) in water (0.26 ml). The mixture was degassed by purging with argon for 10 min. Then was added 1,1′-bis(diphenylphosphino)ferrocene-palladium(II)-dichloride dichloromethane complex (8.8 mg, 0.011 mmol). After purging with argon the tube was sealed and heated to 100° C. for 1.5 h. The mixture was cooled to room temperature and further trimethylboroxine (67 mg, 0.537 mmol) was added and heating at 100° C. was continued for another hour. The residue was treated with aqueous saturated sodium bicarbonate (10 ml) and extracted with ethyl acetate (2×10 ml). The combined organic layers were dried over sodium sulfate, filtered and concentrated in vacuo and the residue was purified by flash column chromatography (silica gel, 0-100% ethyl acetate in heptane) to give tert-butyl N-(4,5-dimethylisothiazol-3-yl)carbamate (20 mg, 40%) as white solid. MS m/z: 173.0 [M−isobutene+H]⁺, ESI pos.

### Step 4: 4,5-dimethylisothiazol-3-amine

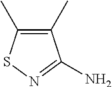

To a solution of tert-butyl N-(4,5-dimethylisothiazol-3-yl)carbamate (24 mg, 0.105 mmol) in dichloromethane (1 ml) was added at 22° C. trifluoroacetic acid (120 mg, 80 ul, 1.05 mmol). The mixture was stirred at 22° C. for 18 h, then it was concentrated in vacuo. The residue was treated with aqueous saturated sodium bicarbonate (10 ml) and extracted with ethyl acetate (2×10 ml). The combined organic layers were dried over sodium sulfate, filtered and concentrated in vacuo to give the title compound (12.5 mg, 93% yield) as light yellow solid. MS m/z: 129.0 [M+H]⁺, ESI pos.

### Intermediate B5: 5-ethyl-4-methyl-isoxazol-3-amine

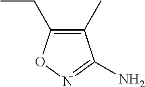

### Step 1: tert-butyl N-(5-ethyl-4-methyl-isoxazol-3-yl)carbamate

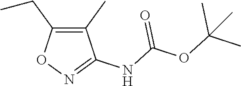 [SMILES: CCc1onc(NC(=O)OC(C)(C)C)c1C]

5-Ethyl-4-methyl-isoxazole-3-carboxylic acid (CAS 1119452-16-2, 208 mg, 1.31 mmol) was dissolved in tert-butanol (2.9 ml). Triethylamine (266 mg, 366 ul, 2.63 mmol) was added, followed by diphenylphosphoryl azide (398 mg, 311 ul, 1.45 mmol) and the resulting solution was heated at 90° C. for 18 h. The solvent was removed under reduced pressure. The resulting material was partitioned between ethyl acetate and water. The layers were extracted and the aqueous layer was extracted twice with ethyl acetate. The combined organic layers were dried over sodium sulfate, filtered and concentrated under reduced pressure. The residue was purified by flash chromatography (silica gel, 0 to 100% ethyl acetate in heptane) to afford tert-butyl N-(5-ethyl-4-methyl-isoxazol-3-yl)carbamate (100 mg, 33%) as white powder. MS m/z: 227.1 [M+H]⁺, ESI pos.

### Step 2: 5-ethyl-4-methyl-isoxazol-3-amine

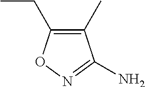

tert-Butyl N-(5-ethyl-4-methyl-isoxazol-3-yl)carbamate (100 mg, 0.433 mmol) was dissolved in 1,2-dichloroethane, extra dry (1 ml) and trifluoroacetic acid (248.64 mg, 168 ul, 2.18 mmol) was carefully added dropwise to the mixture. The resulting solution was stirred at room temperature for 20 h. After completion, a solution of saturated sodium bicarbonate solution was added to the mixture and it was extracted with dichloromethane twice. The combined organic layers were dried over sodium sulfate, filtered and concentrated under reduced pressure to the title compound (47 mg, 81%) as yellow oil which was used without further purification. MS m/z: 127.1 [M+H]⁺, ESI pos.

### Intermediate B6: 4-chloro-5-ethyl-isothiazol-3-amine

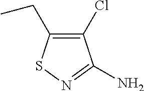

The title compound was prepared in analogy to Intermediate B1 from 4,4,5,5-tetramethyl-2-vinyl-1,3,2-dioxaborolane instead of 4,4,5,5-tetramethyl-2-(prop-1-en-1-yl)-1,3,2-dioxaborolane as white solid, MS (ESI) m/z: 163.0 [M+H]⁺.

### Intermediate B7: 4-ethyl-5-methyl-isothiazol-3-amine

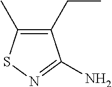

### Step 1: 5-methyl-4-vinyl-isothiazol-3-amine

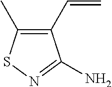

tert-Butyl N-(4-bromo-5-methyl-isothiazol-3-yl)carbamate (see Intermediate B4 Step 2, 300 mg, 0.972 mmol), 4,4,5,5-tetramethyl-2-vinyl-1,3,2-dioxaborolane (195 mg, 1.26 mmol), 1,4-dioxane (4 ml), 2M aqueous sodium carbonate solution (1.46 ml, 2.92 mmol) and 1,1′-bis(diphenylphosphino)ferrocene-palladium(II)dichloride dichloromethane complex (80 mg, 0.097 mmol) were added under argon. The reaction mixture was stirred at 90° C. overnight. After cooling to room temperature, the reaction mixture was poured into water and extracted with ethyl acetate twice. The combined organic layers were washed with brine, dried over sodium sulfate, filtered and concentrated in vacuo. The residue was dissolved in dichloromethane (2 ml), trifluoroacetic acid (1.11 g, 749 ul, 9.72 mmol) was added and the reaction mixture was stirred at room temperature for 18 h. The reaction mixture was poured into saturated sodium bicarbonate solution and extracted with ethyl acetate twice. The combined organic layers were washed with brine, dried over sodium sulfate, filtered and concentrated in vacuo. The crude material was purified by flash chromatography (silica gel, 0 to 50% ethyl acetate in heptane) to give 5-methyl-4-vinyl-isothiazol-3-amine (76 mg, 53% yield) as an off-white solid. MS (ESI) m/z: 141.1 [M+H]⁺.

### Step 2: 4-ethyl-5-methyl-isothiazol-3-amine

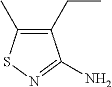

To a solution of (5-methyl-4-vinyl-isothiazol-3-yl)amine (76 mg, 0.542 mmol) in ethyl acetate (20 ml) was added palladium on activated charcoal (58 mg, 0.054 mmol) under argon. The reaction was set under hydrogen atmosphere and the black suspension was stirred at room temperature for 90 min. The reaction mixture was filtered through a pad of dicalite and washed with ethyl acetate. The filtrate was concentrated in vacuo to give the title compound (66 mg, 86% yield) as an off-white semi-solid. MS (ESI) m/z: 143.1 [M+H]⁺.

### Intermediate B8: 4-chloro-5-methoxy-isothiazol-3-amine

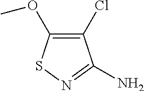

### Step 1: 4-chloro-5-methoxy-isothiazole-3-carboxylic Acid

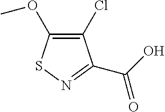

Sodium hydride (60% dispersion in mineral oil, 3.03 g, 75.75 mmol) was carefully added to methanol (105 ml, 2.59 mol) and the mixture was stirred at 25° C. for 30 min. Next, 4,5-dichloroisothiazole-3-carboxylic acid (CAS 131947-13-2, 3.0 g, 15.15 mmol) was added and the mixture was stirred at 80° C. for 6 h. Water (5 ml) was added and the pH of the mixture was adjusted to <7 by addition of 1 N hydrochloric acid. The mixture was extracted with ethyl acetate (3×200 ml) and the combined organic layers was dried over sodium sulfate, filtered and concentrated under reduced pressure. The residue was purified by preparative HPLC (column: Phenomenex luna C18 (250*70 mm, 10 um), water-acetonitrile) to give 4-chloro-5-methoxy-isothiazole-3-carboxylic acid (1.0 g, 34% yield) as an off-white solid. 1H NMR (400 MHz, DMSO-d6) δ=4.16 (s, 3H).

### Step 2: 4-chloro-5-methoxy-isothiazol-3-amine

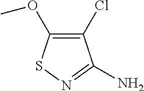

To a solution of 4-chloro-5-methoxy-isothiazole-3-carboxylic acid (300 mg, 1.55 mmol) in tert-butanol (10 ml) was added trimethylamine (188 mg, 1.86 mmol) and diphenylphosphoryl azide (415 mg, 1.7 mmol). The mixture was stirred at 90° C. for 12 h. LCMS showed that the tert-butyl group was already cleaved off. The reaction mixture was concentrated under reduced pressure. The residue was purified by preparative HPLC (column: Phenomenex luna C18 150*25 mm*10 um, water-acetonitrile) to give the title compound (120 mg, 30% yield) as an off-white solid. MS (ESI) m/z: 164.9 [M+H]⁺.

### Intermediate B9: 4-chloro-5-ethoxy-isothiazol-3-amine

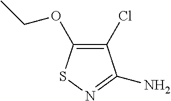

The title compound was prepared in analogy to Intermediate B8 from ethanol instead of methanol. White solid, MS (ESI) m/z: 151.1 [M−Et+H]⁺. 179.1 [M+H]⁺.

### Intermediate B10: 3-(3-amino-4-chloro-isothiazol-5-yl)propanenitrile trifluoroacetate

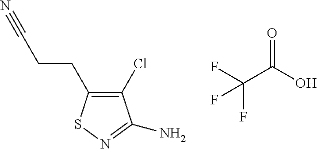 [SMILES: N#CCCc1snc(N)c1Cl ; O=C(O)C(F)(F)F]

### Step 1: 4-chloro-5-(2-cyanoethyl)isothiazole-3-carboxylic Acid Methyl Ester

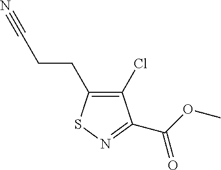

A reaction tube was charged with 4,5-dichloroisothiazole-3-carboxylic acid methyl ester (CAS 166668-76-4, 1.15 g, 5.42 mmol) and tBuXPhos Pd G3 (862 mg, 1.08 mmol). The tube was sealed, degassed and filled with tetrahydrofuran (extra dry, 40 mL) and 2-cyanoethylzinc bromide solution (0.5M in THF, 16.3 mL, 8.13 mmol). The resulting mixture was heated at 60° C. overnight. The reaction mixture was concentrated and purified by flash chromatography (silica gel, ethyl acetate in heptane 0-70%) to afford 4-chloro-5-(2-cyanoethyl)isothiazole-3-carboxylic acid methyl ester (209 mg, 17%) as yellow oil, MS (ESI) m/z: 231.1 [M+H]⁺, ESI pos.

### Step 2: 4-chloro-5-(2-cyanoethyl)isothiazole-3-carboxylic Acid

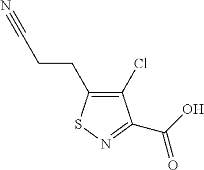 [SMILES: N#CCCc1snc(C(=O)O)c1Cl]

To a solution of 4-chloro-5-(2-cyanoethyl)isothiazole-3-carboxylic acid methyl ester (50 mg, 0.173 mmol) in tetrahydrofuran (1 ml) was added lithium hydroxide solution (1M in water, (260 ul, 0.26 mmol) and the reaction mixture was stirred at room temperature for 30 min. The reaction mixture was acidified with hydrochloric acid (1M in water) and extracted with ethyl acetate twice. The combined organic layers were washed with brine, dried over sodium sulfate and concentrated to afford 4-chloro-5-(2-cyanoethyl)isothiazole-3-carboxylic acid (50 mg, 99%) as light brown oil. MS (ESI) m/z: 217.1 [M+H]⁺, ESI pos.

### Step 3: tert-butyl N-[4-chloro-5-(2-cyanoethyl)isothiazol-3-yl]carbamate

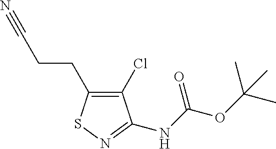 [SMILES: CC(C)(C)OC(=O)Nc1nsc(CCC#N)c1Cl]

To a solution of 4-chloro-5-(2-cyanoethyl)isothiazole-3-carboxylic acid (50 mg, 0.173 mmol) in tert-butanol (1 ml) were added diphenylphosphoryl azide (52 mg, 41 ul, 0.190 mmol) and triethylamine (35 mg, 48 ul, 0.346 mmol) and the reaction mixture was stirred at 90° C. for 2 h. The reaction mixture was diluted with water and extracted with ethyl acetate twice. The combined organic layers were washed with brine, dried over sodium sulfate, filtered and concentrated to dryness. The residue was purified by flash chromatography (silica gel, ethyl acetate in heptane 0-100%) to afford tert-butyl N-[4-chloro-5-(2-cyanoethyl)isothiazol-3-yl]carbamate (19 mg, 38% yield) as colorless oil. MS (ESI) m/z: 286.1 [M−H], ESI neg.

### Step 4: 3-(3-amino-4-chloro-isothiazol-5-yl)propanenitrile Trifluoroacetate

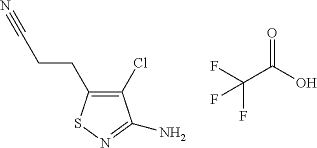 [SMILES: N#CCCc1snc(N)c1Cl ; O=C(O)C(F)(F)F]

To a stirred solution of tert-butyl N-[4-chloro-5-(2-cyanoethyl)isothiazol-3-yl]carbamate (19 mg, 0.066 mmol) in dichloromethane (0.5 ml) was added trifluoroacetic acid (75 mg, 51 ul, 0.66 mmol) and the reaction mixture was stirred at room temperature for 30 min. The reaction mixture was concentrated in vacuo to yield the title compound (20 mg, 100%) as colorless semisolid. MS (ESI) m/z: 188.0 [M+H]⁺, ESI pos.

### Intermediate B11: 5-methylisothiazol-3-amine

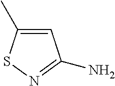

Intermediate B11 is commercial (CAS 128146-85-0).

### Intermediate B12: 4-cyclopropyl-5-methyl-isothiazol-3-amine

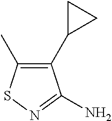

To a solution of tert-butyl N-(4-bromo-5-methyl-isothiazol-3-yl)carbamate (see Intermediate B4 Step 2, 200 mg, 0682 mmol) in toluene (4 ml) was added at room temperature cyclopropylboronic acid (147 mg, 1.71 mmol) followed by potassium phosphate (579 mg, 2.73 mmol) in water (0.8 ml). The mixture was degassed by purging with argon for 10 min. Then was added palladium(II) acetate (7.5 mg, 0.034 mmol) followed by tricyclohexylphosphine (19 mg, 0.068 mmol) and the mixture was again purged with argon. The tube was sealed and heated to 100° C. for 3 h, then the mixture was cooled to 22° C., treated with aqueous sodium carbonate solution (30 ml) and extracted with ethyl acetate (2×30 ml). The combined organic layers were dried over sodium sulfate, filtered and concentrated in vacuo. The residue was purified by flash column chromatography (silica gel, 0-100% ethyl acetate in heptane) to give tert-butyl N-(4-cyclopropyl-5-methyl-isothiazol-3-yl)carbamate (45 mg, 26%) and 4-cyclopropyl-5-methyl-isothiazol-3-amine (51 mg, 48%).

To obtain a second batch of the title compound, tert-butyl N-(4-cyclopropyl-5-methyl-isothiazol-3-yl)carbamate (36 mg, 0.142 mmol) was dissolved in dichloromethane (1.3 ml) and trifluoroacetic acid (162 mg, 108 ul, 1.42 mmol) was added at room temperature and the mixture was stirred overnight. The mixture was concentrated in vacuo and the residue was treated with aqueous saturated sodium bicarbonate solution (10 ml) and extracted with ethyl acetate (2×10 mL). The organic layers were dried over sodium sulfat, filtered and concentrated in vacuo. The residue was purified by flash column chromatography (silica gel, 10-70% ethyl acetate in heptane) to give the title compound (18 mg, 80% yield) as off-white solid. MS (ESI) m/z: 155.0 [M+H]⁺, ESI pos.

### Intermediate B13: 3-amino-5-methyl-isothiazole-4-carbonitrile

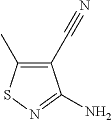

Intermediate B13 is a known compound (CAS 13599-06-9) which is described for example by Hartke, K. et al in Archiv der Pharmazie und Berichte der Deutschen Pharmazeutischen Gesellschaft (1968), 301(8), 611-21.

### Intermediate B14: 4-methoxy-5-methyl-isothiazol-3-amine

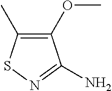

### Step 1: 4-iodo-5-methyl-isothiazole-3-carboxylic Acid

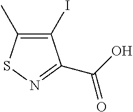

To a stirred solution of 5-methylisothiazole-3-carboxylic acid (CAS 110632-59-2, 500 mg, 3.49 mmol) in trifluoroacetic acid (10 ml) was added N-iodosuccinimide (1.1 g, 4.89 mmol) and the reaction mixture was stirred at 20° C. for 18 h. Water was added and the mixture extracted with ethyl acetate twice. The combined organics were washed with water, dried over sodium sulfate, filtered and concentrated in vacuo to obtain 4-iodo-5-methyl-isothiazole-3-carboxylic acid (383 mg, 41% yield) as off-white solid which was used without further purification. MS (ESI) m/z: 269.8 [M+H]⁺, ESI pos.

### Step 2: 4-methoxy-5-methyl-isothiazole-3-carboxylic Acid

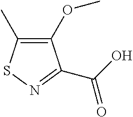

To a solution of sodium methoxide (558 mg, 10.3 mmol) in methanol (50 ml) was added 4-iodo-5-methyl-isothiazole-3-carboxylic acid (556 mg, 2.1 mmol) and copper(I) iodide (118 mg, 0.62 mmol). The reaction mixture was stirred at 80° C. for 48 h, then diluted with water, evaporated and acidified with 1M phosphoric acid to pH 2-3. The aqueous layer was extracted with ethyl acetate (3×) and purified by preparative HPLC (column: Phenomenex luna C18 (250*70 mm, 10 um), acetonitrile/water) to obtain 4-methoxy-5-methyl-isothiazole-3-carboxylic acid (110 mg, 30%) as off-white solid. MS (ESI) m/z: 174.0 [M+H]⁺, ESI pos.

### Step 3: 4-methoxy-5-methyl-isothiazol-3-amine

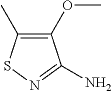

4-Methoxy-5-methyl-isothiazole-3-carboxylic acid (100 mg, 0.58 mmol), diphenylphosphoryl azide (0.12 ml, 0.58 mmol) and triethylamine (0.16 ml, 1.16 mmol) were mixed in tert-butanol (1.0 ml) and heated at 90° C. for 18 h. The reaction mixture was quenched with potassium carbonate solution and extracted with ethyl acetate and evaporated. The residue was dissolved in dichloromethane (0.5 ml), trifluoroacetic acid (0.5 ml) was added and the reaction mixture was stirred at 20° C. for 18 h. The mixture was quenched with diluted aqueous ammonium hydroxide solution and extracted with ethyl acetate twice. The combined organic layers were dried with sodium sulfate, filtered and evaporated to obtain the title compound (22 mg, 26% yield) as colorless oil. MS (ESI) m/z: 145.0 [M+H]⁺, ESI pos.

### Intermediate B15: 4-methoxy-5-propyl-isothiazol-3-amine

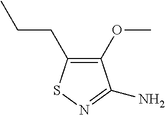

### Step 1: ethyl 5-bromo-4-methoxy-isothiazole-3-carboxylate

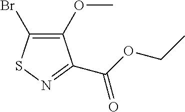

At room temperature, iodomethane (676 mg, 4.76 mmol) were added to a solution of ethyl 5-bromo-4-hydroxy-isothiazole-3-carboxylate (CAS 321601-49-4, described in WO2001014339, 400 mg, 1.59 mmol) and potassium carbonate (658 mg, 4.76 mmol) in dimethylformamide and the reaction mixture was stirred at room temperature for 18 h. After addition of water, the mixture was extracted twice with ethyl acetate. The combined organic phases were washed with water and saturated aqueous sodium chloride solution. Drying over sodium sulfate and removal of the solvent gave ethyl 5-bromo-4-methoxy-isothiazole-3-carboxylate (270 mg, purity 90%, 58% yield). MS (ESI) m/z: 266.0, 268.0 [M+H]⁺, ESI pos.

### Step 2: ethyl 4-methoxy-5-prop-1-enyl-isothiazole-3-carboxylate

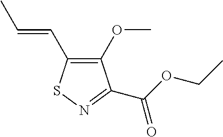

To a mixture of ethyl 5-bromo-4-methoxy-isothiazole-3-carboxylate (206 mg, 0.77 mmol) and 4,4,5,5-tetramethyl-2-(prop-1-en-1-yl)-1,3,2-dioxaborolane (156 mg, 0.93 mmol) in 1,4-dioxane (1 ml) and water (0.25 ml) was added 1,1′-bis(diphenylphosphino)ferrocene-palladium(II)dichloride dichloromethane complex (28.5 mg, 0.03 mmol) and cesium carbonate (507 mg, 1.56 mmol). The reaction mixture was evacuated and re-filled with argon 3 times and stirred at 90° C. for 1.5 h. Then the mixture was cooled and diluted with water (20 ml) and washed with ethyl acetate (80 ml). The combined organic layers were washed with brine (40 ml), dried over sodium sulfate, filtered and concentrated under reduced pressure to give ethyl 4-methoxy-5-prop-1-enyl-isothiazole-3-carboxylate (170 mg, 84% yield), MS (ESI) m/z: 228.0 [M+H]⁺, ESI pos.

### Step 3: ethyl 4-methoxy-5-propyl-isothiazole-3-carboxylate

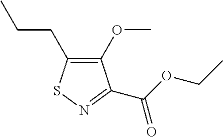

To a solution of ethyl 4-methoxy-5-[(E)-prop-1-enyl]isothiazole-3-carboxylate (170 mg, 0.75 mmol) in methanol (10 ml) was added palladium on charcoal (80 mg) at 20° C. The reaction mixture was stirred under an atmosphere of hydrogen and at 5 bar for 18 h. The reaction mixture was filtered and the solvent was evaporated under reduced pressure to afford ethyl 4-methoxy-5-propyl-isothiazole-3-carboxylate (168 mg, 85% yield), MS (ESI) m/z: 230.0 [M+H]⁺, ESI pos.

### Step 4: 4-methoxy-5-propyl-isothiazole-3-carboxylic Acid

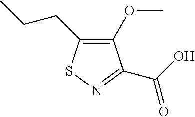

Ethyl 4-methoxy-5-propyl-isothiazole-3-carboxylate (151 mg, 0.66 mmol) and sodium hydroxide (40 mg, 0.99 mmol) were mixed together in ethanol (1.5 ml) and water (0.5 ml) and stirred at 20° C. for 18 h. Next it was evaporated and extracted with methylene chloride (3×10 ml). The organic layers were discarded. The water extract was acidified to pH 4 with phosphoric acid and extracted with ethyl acetate (3×20 ml). The organic extracts were washed with brine (5×20 ml), dried over sodium sulfate, and concentrated under reduced pressure to afford 4-methoxy-5-propyl-isothiazole-3-carboxylic acid (110 mg, 83% yield) as a solid, MS (ESI) m/z: 202.2 [M+H]⁺, ESI pos.

### Step 5: tert-butyl N-(4-methoxy-5-propyl-isothiazol-3-yl)carbamate

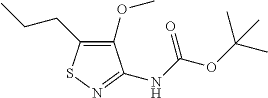 [SMILES: CCCc1snc(NC(=O)OC(C)(C)C)c1OC]

A solution of 4-methoxy-5-propyl-isothiazole-3-carboxylic acid (110 mg, 0.55 mmol), diphenylphosphonic azide (150 mg, 0.12 ml, 0.55 mmol) and triethylamine (111 mg, 0.15 ml, 1.09 mmol) in tert-butanol (0.5 ml) was heated at 90° C. for 18 h. The reaction mixture was quenched with potassium carbonate solution and extracted with ethyl acetate to obtain tert-butyl N-(4-methoxy-5-propyl-isothiazol-3-yl)carbamate (118 mg, 53% yield), MS (ESI) m/z: 217.02 [M−tBu+H]⁺, ESI pos.

### Step 6: 4-methoxy-5-propyl-isothiazol-3-amine

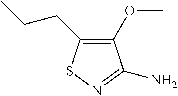

To a stirred solution of tert-butyl N-(4-methoxy-5-propyl-isothiazol-3-yl)carbamate (118 mg, 0.43 mmol) in dichloromethane (0.5 ml) was added trifluoroacetic acid (0.5 ml) and the reaction mixture was stirred at 20° C. for 18 h. Water and ammonium hydroxide solution (25%) were added and the mixture extracted with ethyl acetate. The combined organic layers were washed with water, dried over sodium sulfate, filtered and concentrated in vacuo to obtain the title compound (87 mg, 85% purity, 99% yield) as an oil, MS (ESI) m/z: 173.0 [M+H]⁺, ESI pos.

### Intermediate B16: 4-methyl-5-propyl-isoxazol-3-amine

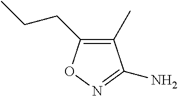

Intermediate B16 is commercial (CAS 1207175-25-4).

### Intermediate B17: 4,5-dimethylisoxazol-3-amine

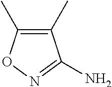

Intermediate B17 is commercial (CAS 13999-39-8).

### Intermediate B18: 4-chloro-5-methyl-isoxazol-3-amine

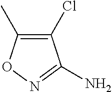

Intermediate B18 is commercial (CAS 5819-39-6).

### Intermediate B19: 4-bromo-5-methyl-isoxazol-3-amine

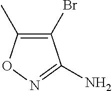

Intermediate B19 is commercial (CAS 5819-40-9).

### EXAMPLES

### Example 1: 6-chloro-N-(4-chloro-5-propyl-isothiazol-3-yl)-1H-indole-3-sulfonamide

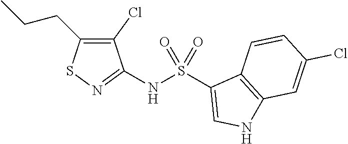 [SMILES: CCCc1snc(NS(=O)(=O)c2c[nH]c3cc(Cl)ccc23)c1Cl]

To a solution of 4-chloro-5-propyl-isothiazol-3-amine hydrochloride (Intermediate B1, 20.5 mg, 0.082 mmol) in dichloromethane, extra dry (0.5 ml) was added diisopropylethylamine (32 mg, 43 ul, 0.245 mmol) followed by 6-chloro-1H-indole-3-sulfonyl chloride (Intermediate A1, 26.5 mg, 0.106 mmol). The reaction mixture was stirred at 23° C. for 2 h, then diluted with water and extracted with dichloromethane (2×7 ml). The combined organic layers were washed with brine, dried over sodium sulfate filtered and concentrated to dryness. The crude compound was purified by preparative HPLC (column: YMC-Triart C18, 12 nm, 5 μm, 100×30 mm, acetonitrile/water+0.1% HCOOH) to afford the title compound (22 mg, 66% yield) as white foam. MS m/z: 390.1[M+H]⁺, ESI pos.

The following Examples 2-4 were prepared in analogy to Example 1 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 2 | 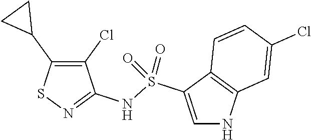 [SMILES: O=S(=O)(Nc1nsc(C2CC2)c1Cl)c1c[nH]c2cc(Cl)ccc12] | 6-chloro-N-(4-chloro-5- cyclopropyl-isothiazol-3- yl)-1H-indole-3- sulfonamide | A1 | B2 | 388.1 [M + H]⁺ |
| 3 | 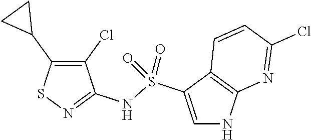 [SMILES: O=S(=O)(Nc1nsc(C2CC2)c1Cl)c1c[nH]c2nc(Cl)ccc12] | 6-chloro-N-(4-chloro-5- cyclopropyl-isothiazol-3- yl)-1H-pyrrolo[2,3- b]pyridine-3-sulfonamide | A2 | B2 | 389.1 [M + H]⁺ |
| 4 | 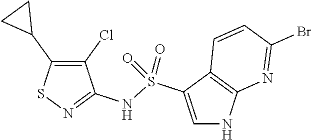 [SMILES: O=S(=O)(Nc1nsc(C2CC2)c1Cl)c1c[nH]c2nc(Br)ccc12] | 6-bromo-N-(4-chloro-5- cyclopropyl-isothiazol-3- yl)-1H-pyrrolo[2,3- b]pyridine-3-sulfonamide | A3 | B2 | 435.1 [M + H]⁺ |

### Example 5: 6-chloro-N-(4-chloro-5-methyl-isothiazol-3-yl)-1H-indole-3-sulfonamide

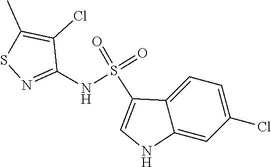 [SMILES: Cc1snc(NS(=O)(=O)c2c[nH]c3cc(Cl)ccc23)c1Cl]

To a solution of 4-chloro-5-methyl-isothiazol-3-amine (Intermediate B3, 26 mg, 0.166 mmol) in dichloromethane, extra dry (0.6 ml) was added diisopropyl ethyl amine (43 mg, 58 ul, 0.332 mmol) and 6-chloro-1H-indole-3-sulfonyl chloride (Intermediate A1, 54 mg, 0.216 mmol,) and the mixture was stirred at room temperature for 45 min. Then it was diluted with ethyl acetate and water and the aqueous phase was extracted with ethyl acetate twice. The combined organic layers were dried over sodium sulfate, filtered and concentrated in vacuo. The residue was purified by flash chromatography (silica gel, methyl acetate in heptane) to give the title compound (21 mg, 35%) as white solid. MS m/z: 362.2 [M+H]⁺, ESI pos.

The following Examples 6-14 were prepared in analogy to Example 5 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 6 | 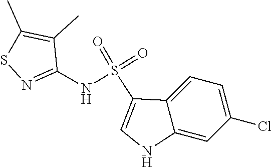 [SMILES: Cc1snc(NS(=O)(=O)c2c[nH]c3cc(Cl)ccc23)c1C] | 6-chloro-N-(4,5- dimethylisothiazol-3-yl)- 1H-indole-3-sulfonamide | A1 | B4 | 342.1 [M + H]⁺ |
| 7 | 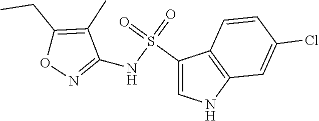 [SMILES: CCc1onc(NS(=O)(=O)c2c[nH]c3cc(Cl)ccc23)c1C] | 6-chloro-N-(5-ethyl-4- methyl-isoxazol-3-yl)-1H- indole-3-sulfonamide | A1 | B5 | 340.1 [M + H]⁺ |
| 8 | 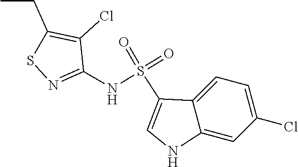 [SMILES: CCc1snc(NS(=O)(=O)c2c[nH]c3cc(Cl)ccc23)c1Cl] | 6-chloro-N-(4-chloro-5- ethyl-isothiazol-3-yl)-1H- indole-3-sulfonamide | A1 | B6 | 376.2 [M + H]⁺ |
| 9 | 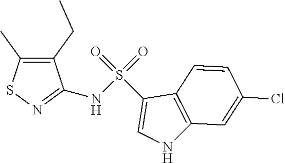 [SMILES: CCc1c(NS(=O)(=O)c2c[nH]c3cc(Cl)ccc23)nsc1C] | 6-chloro-N-(4-ethyl-5- methyl-isothiazol-3-yl)-1H- indole-3-sulfonamide | A1 | B7 | 356.2 [M + H]⁺ |
| 10 | 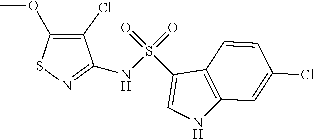 [SMILES: COc1snc(NS(=O)(=O)c2c[nH]c3cc(Cl)ccc23)c1Cl] | 6-chloro-N-(4-chloro-5- methoxy-isothiazol-3-yl)- 1H-indole-3-sulfonamide | A1 | B8 | 378.0 [M + H]⁺ |
| 11 | 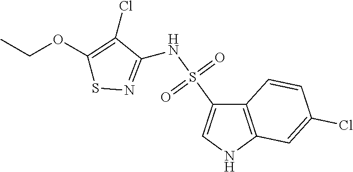 [SMILES: CCOc1snc(NS(=O)(=O)c2c[nH]c3cc(Cl)ccc23)c1Cl] | 6-chloro-N-(4-chloro-5- ethoxy-isothiazol-3-yl)-1H- indole-3-sulfonamide | A1 | B9 | 392.0 [M + H]⁺ |
| 12 | 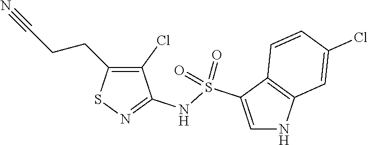 [SMILES: N#CCCc1snc(NS(=O)(=O)c2c[nH]c3cc(Cl)ccc23)c1Cl] | 6-chloro-N-[4-chloro-5-(2- cyanoethyl)isothiazol-3-yl]- 1H-indole-3-sulfonamide | A1 | B10 | 398.9 [M − H]⁻ |
| 13 | 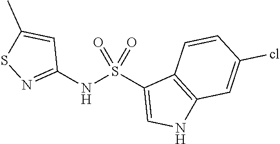 | 6-chloro-N-(5- methylisothiazol-3-yl)-1H- indole-3-sulfonamide | A1 | B11 | 328.0 [M + H]⁺ |
| 14 | 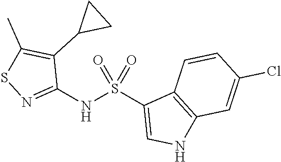 [SMILES: Cc1snc(NS(=O)(=O)c2c[nH]c3cc(Cl)ccc23)c1C1CC1] | 6-chloro-N-(4-cyclopropyl- 5-methyl-isothiazol-3-yl)- 1H-indole-3-sulfonamide | A1 | B12 | 368.2 [M + H]⁺ |

### Example 15: 6-chloro-N-(4-cyano-5-methyl-isothiazol-3-yl)-1H-indole-3-sulfonamide

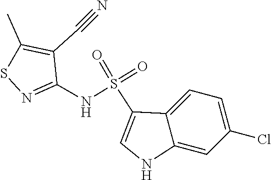 [SMILES: Cc1snc(NS(=O)(=O)c2c[nH]c3cc(Cl)ccc23)c1C#N]

To a mixture of 3-amino-5-methyl-isothiazole-4-carbonitrile (Intermediate B13, 25 mg, 0.180 mmol) and 6-chloro-1H-indole-3-sulfonyl chloride (Intermediate A1, 57 mg, 0.216 mmol) was added pyridine (800 ul). The reaction mixture was stirred at room temperature for 1 h, then the solution was concentrated in vacuo. The crude material was purified by flash chromatography (silica gel, 0-60% ethyl acetate in heptane), followed by preparative HPLC (column: YMC-Triart C18, 12 nm, 5 urn, 100×30 mm, acetonitrile/water+0.1% HCOOH) to afford the title compound (12 mg, 19%) as white solid. MS (ESI) m/z: 353.0 [M+H]⁺.

The following Examples 16-17 were prepared in analogy to Example 15 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 16 | 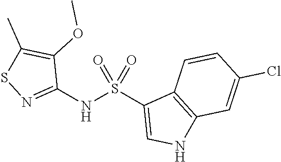 [SMILES: COc1c(NS(=O)(=O)c2c[nH]c3cc(Cl)ccc23)nsc1C] | 6-chloro-N-(4-methoxy-5- methyl-isothiazol-3-yl)-1H- indole-3-sulfonamide | A1 | B14 | 358.0 [M + H]⁺ |
| 17 | 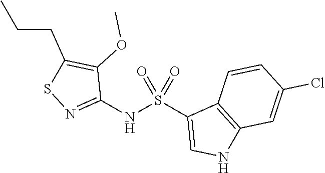 [SMILES: CCCc1snc(NS(=O)(=O)c2c[nH]c3cc(Cl)ccc23)c1OC] | 6-chloro-N-(4-methoxy-5- propyl-isothiazol-3-yl)-1H- indole-3-sulfonamide | A1 | B15 | 386.0 [M + H]⁺ |

### Example 18: 6-chloro-N-(4-methyl-5-propyl-isoxazol-3-yl)-1H-indole-3-sulfonamide

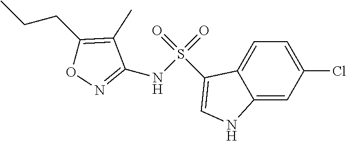 [SMILES: CCCc1onc(NS(=O)(=O)c2c[nH]c3cc(Cl)ccc23)c1C]

To a solution of 4-methyl-5-propyl-isoxazol-3-amine (Intermediate B16, 38 mg, 0.27 mmol) and N,N-diisopropyl ethyl amine (52 mg, 0.07 ml, 0.41 mmol) in dry pyridine (1 ml) was added 6-chloro-1H-indole-3-sulfonyl chloride (Intermediate A1, 75 mg, 0.3 mmol) and mixture was stirred at room temperature for 18 h. Then mixture was purified by preparative HPLC (column: YMC Triart C18, 100×20 mm, 5 um, water/acetonitrile+0.10% NH4OH) to give the title compound (8 mg, 8% yield) as yellow solid. MS (ESI) m/z: 354.0 [M+H]⁺.

### Example 19: 6-chloro-N-(4,5-dimethylisoxazol-3-yl)-1H-indole-3-sulfonamide

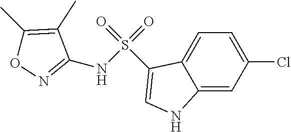 [SMILES: Cc1onc(NS(=O)(=O)c2c[nH]c3cc(Cl)ccc23)c1C]

The title compound was prepared in analogy to Example 18 from Intermediate B17 instead of Intermediate B16. Orange solid, MS (ESI) m/z: 326.0 [M+H]⁺.

### Example 20: 6-chloro-N-(4-chloro-5-methyl-isoxazol-3-yl)-1H-indole-3-sulfonamide

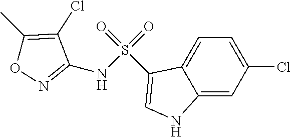 [SMILES: Cc1onc(NS(=O)(=O)c2c[nH]c3cc(Cl)ccc23)c1Cl]

4-Chloro-5-methyl-isoxazol-3-amine (Intermediate B18, 40 mg, 0.30 mmol), 6-chloro-1H-indole-3-sulfonyl chloride (Intermediate A1, 50 mg, 0.20 mmol) and 4-dimethylaminopyridine (2.5 mg, 0.02 mmol) were introduced in a reaction vial. Pyridine (extra dry, 3 ml) was added at room temperature and the mixture was stirred for at 100° C. for 1 h. The reaction mixture was cooled to room temperature, diluted with citric acid (1M in water) and extracted two times with ethyl acetate. The combined organic layers were washed with water and brine, dried over sodium sulfate and concentrated to dryness. The residue was purified by preparative HPLC (column: YMC-Triart C18, 12 nm, 5 μm, 100×30 mm, acetonitrile/water+0.1% HCOOH) to afford the title compound (19 mg, 27% yield) as white solid, MS (ESI) m/z: 346.1 [M+H]⁺.

### Example 21: N-(4-bromo-5-methyl-isoxazol-3-yl)-6-chloro-1H-indole-3-sulfonamide

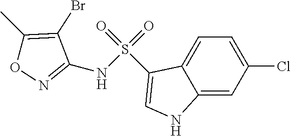 [SMILES: Cc1onc(NS(=O)(=O)c2c[nH]c3cc(Cl)ccc23)c1Br]

The title compound was prepared in analogy to Example 20 from Intermediate B19 instead of Intermediate B18. White solid, MS (ESI) m/z: 391.9 [M+H]⁺.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. Compounds of formula I 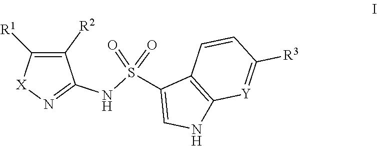
wherein,
R¹ is alkyl, alkoxy, cyano, cyanoalkyl, cyclopropyl, or halo;
R² is H, alkyl, alkoxy, cyano, cyclopropyl or halo;
R³ is halo, alkyl, or haloalkyl;
X is —O— or —S—;
Y is CH or N;
and pharmaceutically acceptable salts thereof.

2. A compound according to claim 1, wherein R¹ is alkyl, alkoxy, cyanoalkyl or cyclopropyl.

3. A compound according to claim 1, wherein R² is H, alkyl, alkoxy, cyano, cyclopropyl or halo.

4. A compound according to claim 1, wherein R² is alkoxy or halo.

5. A compound according to claim 1, wherein R³ is halo or alkyl.

6. A compound according to claim 1, wherein R³ is halo.

7. A compound according to claim 1, wherein X is —S—.

8. A compound according to claim 1 wherein
R¹ is alkyl, alkoxy, cyanoalkyl, or cyclopropyl;
R² is H, alkyl, alkoxy, cyano, cyclopropyl or halo;
R³ is halo;
X is —O— or —S—;
Y is CH or N;
and pharmaceutically acceptable salts thereof.

9. A compound according to claim 1 wherein
R¹ is alkyl, alkoxy, cyanoalkyl, or cyclopropyl;
R² is alkoxy or halo;
R³ is halo;
X is —S—;
Y is CH or N;
and pharmaceutically acceptable salts thereof.

10. A compound according to claim 1, selected from
6-chloro-N-(4-chloro-5-propyl-isothiazol-3-yl)-1H-indole-3-sulfonamide;
6-chloro-N-(4-chloro-5-cyclopropyl-isothiazol-3-yl)-1H-indole-3-sulfonamide;
6-chloro-N-(4-chloro-5-cyclopropyl-isothiazol-3-yl)-1H-pyrrolo[2,3-b]pyridine-3-sulfonamide;
6-bromo-N-(4-chloro-5-cyclopropyl-isothiazol-3-yl)-1H-pyrrolo[2,3-b]pyridine-3-sulfonamide;
6-chloro-N-(4-chloro-5-methyl-isothiazol-3-yl)-1H-indole-3-sulfonamide;
6-chloro-N-(4,5-dimethylisothiazol-3-yl)-1H-indole-3-sulfonamide;
6-chloro-N-(5-ethyl-4-methyl-isoxazol-3-yl)-1H-indole-3-sulfonamide;
6-chloro-N-(4-chloro-5-ethyl-isothiazol-3-yl)-1H-indole-3-sulfonamide;
6-chloro-N-(4-ethyl-5-methyl-isothiazol-3-yl)-1H-indole-3-sulfonamide;
6-chloro-N-(4-chloro-5-methoxy-isothiazol-3-yl)-1H-indole-3-sulfonamide;
6-chloro-N-(4-chloro-5-ethoxy-isothiazol-3-yl)-1H-indole-3-sulfonamide;
6-chloro-N-[4-chloro-5-(2-cyanoethyl)isothiazol-3-yl]-1H-indole-3-sulfonamide;
6-chloro-N-(5-methylisothiazol-3-yl)-1H-indole-3-sulfonamide;
6-chloro-N-(4-cyclopropyl-5-methyl-isothiazol-3-yl)-1H-indole-3-sulfonamide;
6-chloro-N-(4-cyano-5-methyl-isothiazol-3-yl)-1H-indole-3-sulfonamide;
6-chloro-N-(4-methoxy-5-methyl-isothiazol-3-yl)-1H-indole-3-sulfonamide;
6-chloro-N-(4-methoxy-5-propyl-isothiazol-3-yl)-1H-indole-3-sulfonamide;
6-chloro-N-(4-methyl-5-propyl-isoxazol-3-yl)-1H-indole-3-sulfonamide;
6-chloro-N-(4,5-dimethylisoxazol-3-yl)-1H-indole-3-sulfonamide;
6-chloro-N-(4-chloro-5-methyl-isoxazol-3-yl)-1H-indole-3-sulfonamide;
N-(4-bromo-5-methyl-isoxazol-3-yl)-6-chloro-1H-indole-3-sulfonamide;
and pharmaceutically acceptable salts thereof.

11. A compound according to claim 1, selected from
6-chloro-N-(4-chloro-5-propyl-isothiazol-3-yl)-1H-indole-3-sulfonamide;
6-chloro-N-(4-chloro-5-cyclopropyl-isothiazol-3-yl)-1H-indole-3-sulfonamide;
6-bromo-N-(4-chloro-5-cyclopropyl-isothiazol-3-yl)-1H-pyrrolo[2,3-b]pyridine-3-sulfonamide;
6-chloro-N-(4-chloro-5-methyl-isothiazol-3-yl)-1H-indole-3-sulfonamide;
6-chloro-N-(4-chloro-5-ethyl-isothiazol-3-yl)-1H-indole-3-sulfonamide;
6-chloro-N-(4-chloro-5-ethoxy-isothiazol-3-yl)-1H-indole-3-sulfonamide;
6-chloro-N-[4-chloro-5-(2-cyanoethyl)isothiazol-3-yl]-1H-indole-3-sulfonamide;
6-chloro-N-(4-methoxy-5-propyl-isothiazol-3-yl)-1H-indole-3-sulfonamide;
and pharmaceutically acceptable salts thereof.

12. A process to prepare a compound according to claim 1 comprising the reacting a compound of formula III with a compound of formula II in the presence of a base selected from N-ethyldiisopropylamine or pyridine with or without the addition of the catalyst 4-dimethylaminopyridine, or in the presence of an inorganic base like potassium phosphate, to provide a compound of formula I, 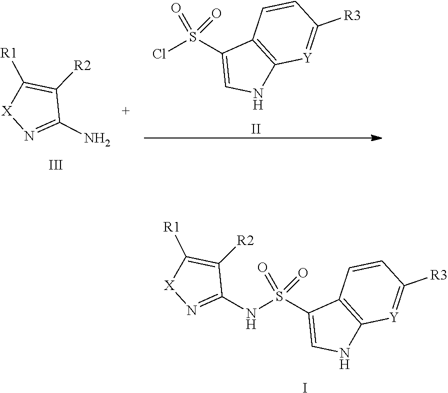
wherein R¹, R², R³, X and Y are as described above.

13-14. (canceled)

15. A pharmaceutical composition comprising a compound according to claim 1 and a therapeutically inert carrier.

16-20. (canceled)

21. A method for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and inflammation in the CNS for instance following encephalitis, primary angiitis, meningitis and obesity, which method comprises administering an effective amount of a compound of claim 1 to a patient in need thereof.

22. A method for the treatment or prophylaxis of multiple sclerosis, which method comprises administering an effective amount of a compound of claim 1 to a patient in need thereof.

23-24. (canceled)
